(19) United States
(12) Patent Application Publication
(10) Pub. No.: US 2003/0181898 A1
(43) Pub. Date: Sep. 25, 2003

(54) RF filter for an electrosurgical generator

(76) Inventor: William J. Bowers, Highlands Ranch, CO (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/376,724

(22) Filed: Feb. 28, 2003

Related U.S. Application Data
(63) Continuation of application No. 09/579,355, filed on May 26, 2000, now abandoned.
(60) Provisional application No. 60/137,125, filed on May 28, 1999.

(51) Int. Cl. A61B 18/12

(52) U.S. Cl. 606/34; 606/38; 606/39; 606/40

(57) ABSTRACT

Disclosed is an electrosurgical generator that comprises a circuit and method to produce a filtering technique that utilizes the output transformer as the inductive element of the filter and places two capacitors on the output of the transformer to produce the desired filtering. The output filter configuration eliminates the use of power inductors and still creates the filtering required in electrosurgical applications. The filter utilizes the inductive properties of the output transformer along with several capacitors to form the bandpass filter.

### CROSS REFERENCE TO RELATED APPLICATION

The present invention claims the benefit of earlier-filed U.S. provisional patent application, serial No. 60/137,125, filed on May 28, 1999, which is hereby incorporated by reference in its entirety herein.

### BACKGROUND OF THE INVENTION

1. Field of the Invention

The present invention relates to an electrosurgical generator, and more specifically, to an RF filter for an electrosurgical generator.

2. Background Information

The use of RF energy to cut and coagulate is well known. Many different circuits have been developed to produce safe and effective electrosurgical energy. It has been found that in order to produce effective electrosurgical energy while maintaining a high level of safety, the electrosurgical generator should produce a “clean” sinusoidal waveform.

It is also known that the most efficient method of creating RF energy is to use a power amplifier that is operating in a class D configuration. This means that all of the power devices are switching on and off into a saturation mode. The faster the power components switch, within components limits, the more efficient the system will become. The problem with class D operation is that the output waveform resembles a square wave. This square wave is made up of the fundamental frequency along with many high frequencies. If these high frequencies are allowed to go out to the operating site, a large amount of RF leakage can be produced. To resolve the problem of the square wave, a bandpass filter is used to filter out the high and low frequencies and pass the fundamental frequency to the output. In general, such a bandpass filter consists of power inductors. The cost of the inductors used in a high powered application, however, can be costly in both material and labor. Thus a system that eliminates the cost of these high price devices can be advantageous over the prior art.

### SUMMARY OF THE INVENTION

Disclosed is an electrosurgical generator that comprises a circuit and method to produce a filtering technique that utilizes the output transformer as the inductive element of the filter and places two capacitors on the output of the transformer to produce the desired filtering that addresses the shortcomings of the prior art. Disclosed is a new output filter configuration that eliminates the use of power inductors and still creates the filtering required in electrosurgical applications. The filter utilizes the inductive properties of the output transformer along with several capacitors to form the bandpass filter. For illustrative purposes only, the invention is disclosed in conjunction with a bipolar output circuitry, but may also be used in a monopolar application.

### BRIEF DESCRIPTION OF THE DRAWINGS

FIG. 1 illustrates a typical frequency response of a bandpass filter;

FIG. 2 illustrates a circuit simulation used for producing the frequency response of the FIG. 1;

FIG. 3 illustrates a transient response to a 500 kHz square wave input;

FIG. 4 illustrates a transformer circuit diagram;

FIG. 5 illustrates an equivalent high frequency transformer;

FIG. 6 illustrates an equivalent second ordered low pass filter;

FIG. 7 illustrates a frequency response of the filter of FIG. 6;

FIG. 8 illustrates an equivalent second ordered low pass filter with a 1000 pFd capacitor added to the output;

FIG. 9 illustrates a frequency response of the filter of FIG. 8;

FIG. 10 illustrates an equivalent second-ordered low pass filter with 1000 pFd capacitor and mutual inductance of the output transformer;

FIG. 11 illustrates a frequency response to the equivalent second ordered low pass filter with 1000 pFd capacitor and mutual inductance of the output transformer of FIG. 10;

FIG. 12 illustrates a final filter configuration producing bandpass characteristics;

FIG. 13 illustrates a frequency response for the final filter configuration producing bandpass characteristics;

FIG. 14 illustrates a transient response to a 500 kHz square wave input;

FIG. 15 illustrates a transformer circuit with external capacitors producing bandpass characteristics;

FIG. 16 illustrates a frequency response of the transformer circuit with external capacitors producing bandpass characteristics illustrated in FIG. 15;

FIGS. 17a and 17b illustrate transient response to a 500 kHz square wave input;

FIG. 18 is a general block diagram of the power and logic sections of the electrosurgical generator;

FIG. 19 is a load curve graphing output power to output load impedance of the electrosurgical generator; and

FIG. 20 illustrates typical RF logic waveform.

### DESCRIPTION OF THE PREFERRED EMBODIMENT

Before explaining the present invention in detail, it should be noted that the invention is not limited in its application or use to the details of construction and arrangement of parts illustrated in the accompanying drawings and description, because the illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

I. General Electrosurgical Operation

System Operation

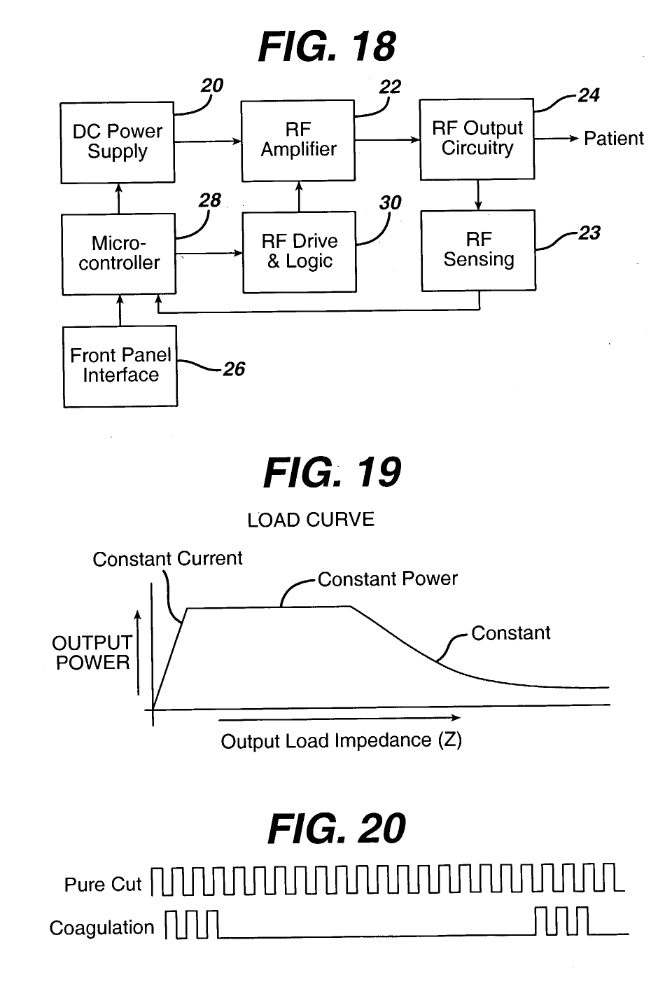

In order to understand the operation of the invention a general description of the power control system operation of an electrosurgical generator is required. FIG. 18 shows the main functions that describe the general operation of an electrosurgical generator. Additional block diagram functions are required to explain the complete operation of an electrosurgical generator but are not included in this disclosure, but are well known to those skilled in the art.

DC Power Supply

The DC power supply 20 converts AC line voltage to a DC voltage that drives the RF amplifier 22. The power supply 20 regulates the output current, power and voltage. (Prior art shows that the same regulation properties can be achieved by a Pulse Width Modulation (PWM) technique on a RF amplifier. This disclosure does not use this PWM technique, but the algorithm described within will operate using the PWM technique). As the RF energy is delivered, the power supply 20 determines the appropriate regulation mode (current, power or voltage) in which it should operate. The graph of FIG. 19 provides a better understanding of the regulation modes.

Referring to FIG. 19, constant current occurs at the lower impedances when the output requires higher current than a pre-set value. The different modes of operation, such as cut or coagulation, require different constant current limits. As long as the output impedance is low the DC power supply 20 will regulate to the constant current. When the output impedance increases, the DC power supply 20 will change to a constant power regulation. Constant power operates within the medium range of impedances and limits the output power for each power setting.

This constant power allows the same amount of power to be delivered to the patient as long as the impedance is within the proper range. As the output impedance continues to increase, the DC power supply 20 begins to regulate with a constant voltage. The constant voltage limits the output DC voltage that is delivered to the RF amplifier 22. In turn, the output power reduces in an inverse relationship (1/Z) to the output impedance. At the point in which the system changes from one regulation mode to the next, a “roll-off” point can be defined. For example, if the Cut mode transitions from the power regulation mode to the voltage regulation mode at 800 ohms, the cut mode is said to have an upper “roll-off” point of 800 ohms.

RF Amplifier

The RF amplifier 22 converts the DC voltage to a high frequency, energy. Any type of RF amplifier can be used to create the electrosurgical energy, i.e., Full Bridge, Single Ended, Push-Pull, etc. By controlling the RF energy, as described in the DC power supply 20 section and by controlling the output waveform, different clinical effects are produced. The cut mode is produced by creating a continuous, approximately 500 kHz RF waveform. Applying a duty cycle to the waveforms produces Blend (Cutting with Hemostasis), Coagulation (Fulguration and Contact) and Bipolar Coagulation modes. Different clinical effects can be achieved depending on the duty cycle and the “roll-off” points of the mode of operation.

RF Output Circuitry

The RF output circuitry 24 begins with the output transformer and ends at the operating site. All RF amplifiers have an output transformer to convert the low primary voltage to a higher output voltage. The cut modes require less voltage, but higher power levels than the coagulation modes. The output transformer steps the primary voltage to a point where the requirements of the roll-off points at maximum power settings are met. In the coagulation mode, especially in the Spray or Fulguration mode, the turns ratio is much higher than in the cut modes. This allows for a very high voltage level when the generator is operated into a high impedance in the voltage regulation mode. The high voltage is required for creating and maintaining arcs to the operating site.

In most cases, the RF output circuitry 24 requires a high frequency filter. (Many Single Ended type amplifiers operate into a “tune tank”, thus producing sinusoidal waveforms naturally.) The filter coverts the input waveform, basically a square wave, into a sinusoidal waveform. By converting the output to a sinusoidal waveform, the problem with RF leakage is reduced. In addition, the RF output circuitry 24 requires relays to direct the RF energy to the appropriate output accessory and, in turn, to the operative site.

RF Sensing

Many electrosurgical generators utilize a method of sensing the output RF energy delivered to the patient. Other electrosurgical generators sense the DC Power Supply to determine the energy delivered to the patient via the RF amplifier. The present generator senses the DC Power Supply but, in addition, senses the output of the RF generator through an RF sensing circuit 23, which comprises a Voltage Peak Detector and a Current Sense Detector as disclosed in U.S. patent application Ser. No. ______, docket number END-643 filed concurrently herewith and incorporated by reference herein.

Front Panel Interface

The Front Panel Interface 26 is simply the interface between the operating room personnel and the generator. The operating room personnel instruct the generator of the appropriate mode and power setting for the clinical procedure. In turn, the front panel displays the requested information and provides activation information to the operating room personnel. In most generators, when the operating room personnel activate a mode, the front panel initiates an indicator to inform the user which mode is operated.

RF Drive & Logic

Electrosurgical generators develop the RF drive signals many different ways. Some use discrete transistors, some use logic systems, while others use a combination of microcontroller and logic to determine the RF drive pulses. In all electrosurgical generators different waveforms are generated for given modes of operations by the RF drive and logic circuit 30. For example, a pure cut waveform would have continuous train of pulses delivered to the RF amplifier 22. A coagulation waveform would have a short duration of pulses (in some cases only one pulse) and a long duration of off time, which provides high peak energy while producing low RMS power to the surgical site required for coagulation. The train of pulses will operate at the operating frequency of the RF amplifier 22. All electrosurgical generators operate between 300 kHz and 5 MHz, with the majority operating around 500 kHz. Typical RF logic waveforms are shown in FIG. 20.

The RF drive receives pulses from the RF logic and increases the energy required to operate the RF amplifier 22. For example, the power MOSFETs used in the RF amplifier 22 requires a switching signal that is positive 15 volts to properly turn the device on and off. The RF drive and logic circuit 30 produces these required voltage levels. Many different circuits are well known to properly operate the power MOSFET transistors inside the RF amplifier.

Microcontroller

The microcontroller 28 is the heart of the system. (In some simple electrosurgical generators, a microcontroller is not used. Instead, logic systems are used to determine the way the system is operating). Its main function is to determine the system operation depending on the input received. In addition, the microcontroller 28 monitors the safety features of the system. If a safety system shows a problem, the microcontroller 28 determines and implements the appropriate action.

The microcontroller 28 determines the level of DC voltage required to produce a given mode of operation, RF drive waveforms, “roll-off” points to determine the load curve, and many other functions of the generator. By using a microcontroller 28, many functions, input and output, can be conducted simultaneously. For example, when the generator is activated in the Spray Coag mode, an input to the microcontroller 28 can monitor the peak voltage of the output waveform. If the output voltage reaches a pre-determined level the RF drive signal could be modified. By adjusting the RF drive waveform, different physiological effects can be obtained.

II. RF Filter for Electrosurgical Generator

RF filters are used for many different functions, such as passing low and/or high frequencies, blocking low and/or high frequencies and phase correction, to name a few. In the case of electrosurgical generators, filters are used to pass the operating frequency and block the unwanted frequencies from being delivered to the patient. In the prior art, many different types of filters and locations of the filters are used. For example, many different electrosurgical generators use a bandpass filter scheme on the output of the output transformer. These filters will block both the low and high frequencies while passing the fundamental frequency. Since the output powers are very high in the electrosurgical generators, all output filters use inductors and capacitors that can handle these high levels of powers.

Filtering Characteristics

The filter needs to have certain characteristics in order to operate properly in the electrosurgical generator. Specifically, the filter needs to pass the fundamental frequency to the operating site while blocking all other high and low frequencies. These characteristics are typical of a bandpass filter.

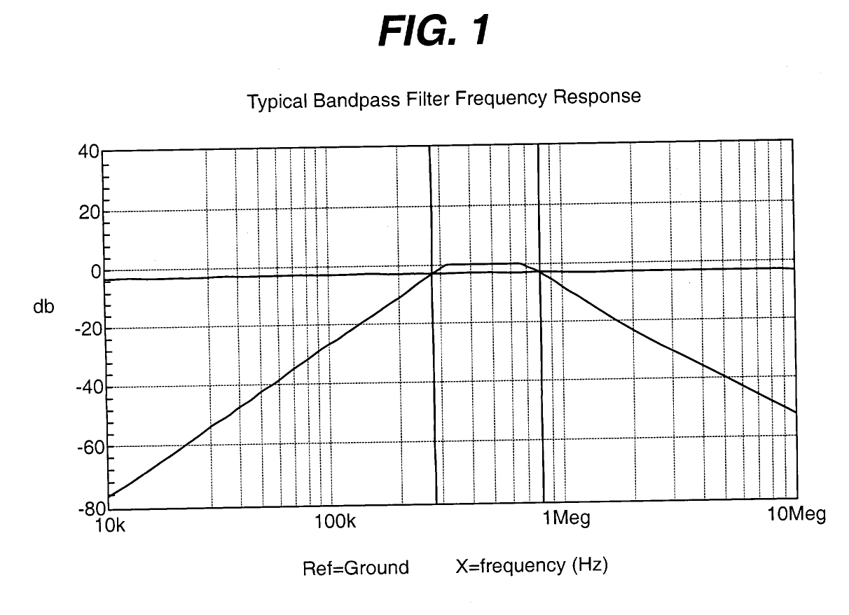

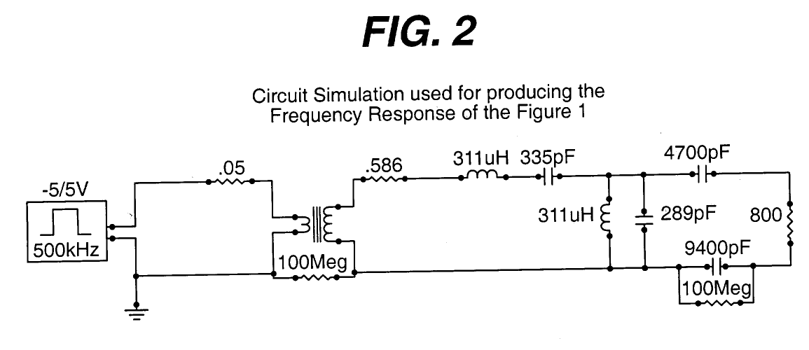

FIG. 1 is an example of a frequency response of a bandpass filter. A circuit analysis program was used to produce the frequency response from the circuit provided in FIG. 2. The waveform shows the gain of the circuit as a function of frequency. The Y-axis shows the gain stated in dB levels (+dB indicates a positive gain in the system while −dB indicates attenuation). The X-axis shows the frequency as the independent variable and is provided in a log scale.

As can be seen in FIG. 1, the frequencies around 500 kHz are passed while both the low and high frequencies are attenuated. Bandwidth, the group of frequencies that pass through a bandpass filter, is defined as the frequencies that are above the −3 db frequencies. By observing the graph in FIG. 1, the two frequencies that cross the −3 db points are approximately 799 kHz and 274 kHz. By subtracting the lower frequency from the higher frequency the bandwidth of the filter in FIG. 2 can be determined. In this case the bandwidth is approximately 525 kHz (799 kHz−274 kHz=525 kHz). Stating this another way; all frequencies between 274 kHz and 799 kHz are passed to the output load while all frequencies outside of these parameters are considered to be in the stop bands and are stopped from going to the output load. As a result, a square wave waveform operating at approximately 500 kHz, subjected to the filter would produce a sinusoidal waveform since the fundamental frequency would pass and all high and low frequencies will be stopped.

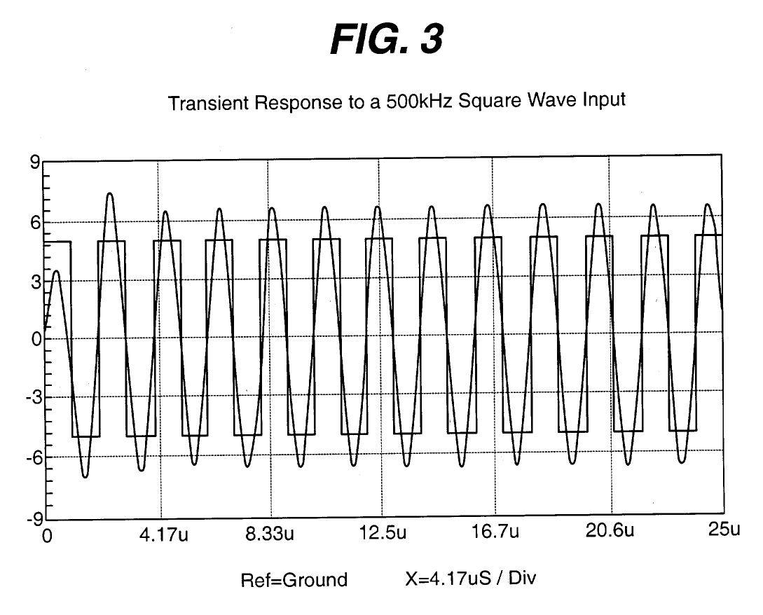

A simulation of the output waveform when the bandpass filter is subjected to a 500 kHz square wave is provided in FIG. 3. This simulation shows the input waveform and the output waveform on the same graph. The results show a “clean” sine wave being produced from the square wave input. The goal of this disclosure is to simulate the characteristics of the bandpass filter but use only the output transformer and a few low cost capacitors.

Output Transformer

Output transformers used in electrosurgical generators are subjected to high frequencies and high power. This makes them difficult to design since these two parameters conflict with each other. Special materials and winding techniques are required to develop a good operating transformer.

The type of transformer used in the present electrosurgical generator is a toroidal configuration (a round, “doughnut” shape core), though any other type of core configuration may work to practice this invention. The winding of the transformer is done in such a way as to reduce the amount of leakage inductance and stray capacitance, which is well known to those skilled in the art.

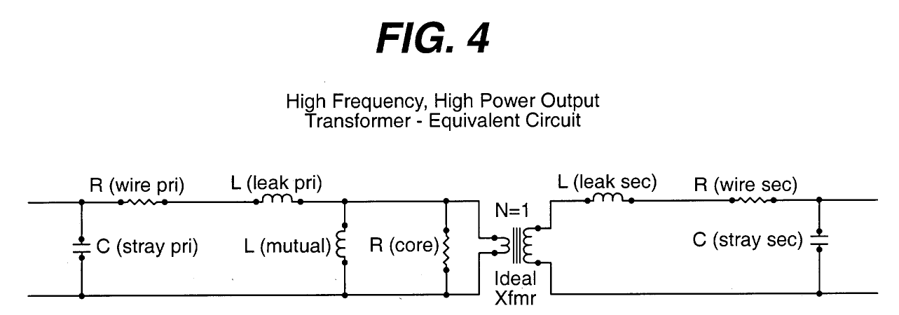

Referring to FIG. 4, the circuit of a high frequency, high power transformer has many equivalent components that can affect the output characteristics of the output transformer. But after analysis it can be shown that only a few of the components affect the output, if the transformer is designed properly. Each equivalent component is briefly described below keeping in mind that the main function of a transformer is to step up or step down the input voltage while isolating the primary circuits from the output circuits.

Ideal Transformer (Xfmr)

The ideal transformer (Xfmr), in the middle of the equivalent circuit, has no losses (all losses are represented by the other equivalent components). The turns ratio, N:1, either steps up the input voltage or steps down the input voltage. In the majority of electrosurgical generators currently on the market, the transformers are used in a step up configuration; therefore, the N is less than one (depending on the turns ratio). For example, if the primary turns were 30 and the secondary turns were 75 then N would be 0.4. In Bipolar applications, the turns ratio is usually closer to one. Throughout the rest of this disclosure the turns ratio will be assumed to be one. This greatly simplifies the calculations for analyzing the final equivalent circuit.

C_{(stray pri)} and C_{(stray sec)}

C_{(stray pri)} is the stray capacitance that is developed by the primary windings. Since the wires are wound close together a capacitance is developed in parallel with the primary windings. C_{(stray sec)} is the stray capacitance that is developed by the secondary windings. Once again, as in the primary stray capacitance, the wires are wound close together and a capacitance is developed in parallel with the secondary windings. It should be noted that both the stray capacitances are shown just on the input and the output of the equivalent circuit but in reality the stray capacitance is distributed throughout the transformer. Since the capacitance is distributed it can be shown that both the primary and secondary capacitances can be combined as one capacitor on the output of the transformer. This value is very low but still plays a roll in how the transformer reacts to high frequencies. This will be shown later.

R_{(wire pri)} and R_{(wire sec)}

This is the resistance of the wire that is used to wind the primary and secondary windings. In both cases, the current in electrosurgical applications is relatively low, therefore the resistive effect is negligible. This is assuming that the designer applies appropriate wire sizes to both of these wires. Both of these resistances can be eliminated from our analysis.

L_{(leak pri)} and L_{(leak sec)}

L_{(leak pri)} is the leakage inductance that is developed by the primary windings and L_{(leak sec)} is the leakage inductance that is developed by the secondary windings. Leakage inductances are the lines of flux that are not coupled to or from the magnetic material and are considered a loss (reactive loss) that must be overcome. If the leakage inductance is high and the transformer operates in a high frequency application, as in electrosurgical applications, the leakage inductance becomes a major design factor in the development of the transformer.

Since both leakage inductances are in series, then both inductances can be combined together through the turns ratio of the ideal transformer. Since our example uses a turns ratio of one then the two inductances are just additive.

R_{(core)}

R_{(core)} represents the resistive (real) power loss of the core when subjected to an input voltage and frequency. Since high quality core materials are now being used in the development of output transformers for electrosurgical applications, this loss is relatively low. Though there is some heating in the output transformer when the electrosurgical generator is operated in an open circuit mode, it is not enough to vary the effect of the model and will be eliminated from our model. It should be noted that core loss would provide some damping in the final filtering to the output waveforms.

L₍ₘᵤₜᵤₐₗ₎

This is the mutual inductance that is shared between the primary and the secondary windings. The value is determined by the type and size of core used in the application and the number of turns wound on the core. Within limits, this value can vary widely and is determined by the final design of the transformer. As will be seen later, this value will be adjusted depending upon the desired bandpass characteristics.

Equivalent High Frequency Transformer

By combining all of the assumptions outlined above, an equivalent high frequency transformer can be developed. Keep in mind that the model is simplified further because of the one-to-one turns ratio of the transformer. If a turns ratio other than one is used, the same model can apply but the values in the equivalent model change greatly because of the turns squared relationship of the resistances and the reactances of the primary and secondary circuits as is readily apparent to those skilled in the art.

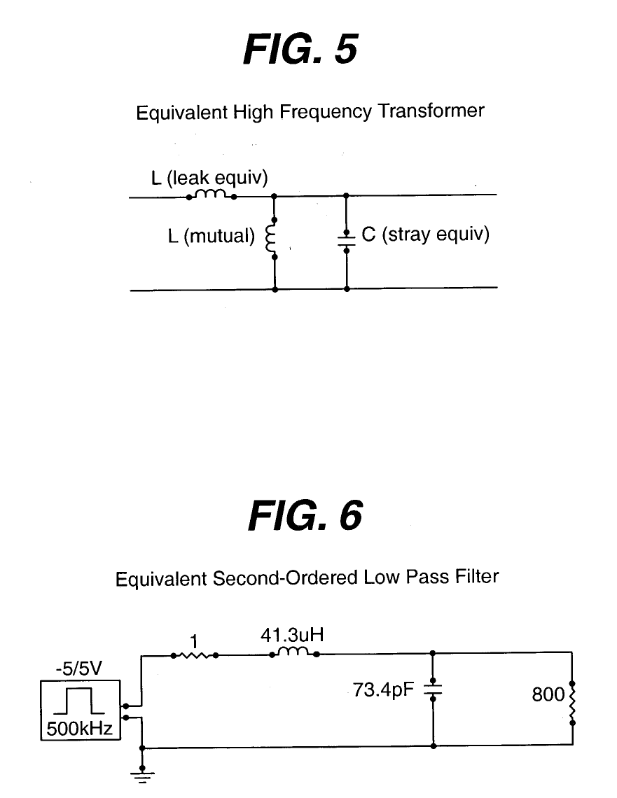

Referring to FIG. 5, the output transformer reduces down to a simple equivalent circuit. To further simplify the circuit, if the L₍ₘᵤₜᵤₐₗ₎ has a high value of inductance then the reactance of the mutual inductance would be high for the frequency used in electrosurgery. As seen later in this disclosure, the value of inductance will be picked to provide attenuation for the lower frequencies. For the moment, let's remove L₍ₘᵤₜᵤₐₗ₎ from the model and look at the resultant circuit.

By removing the L₍ₘᵤₜᵤₐₗ₎ from the equivalent high frequency transformer we are left with a simple second-ordered low pass filter. Through experimentation of the one-to-one transformer, the values of the L_{(leak equiv)} and C_{(stray equiv)} are utilized in the equivalent second-ordered low pass filter outlined in FIG. 6.

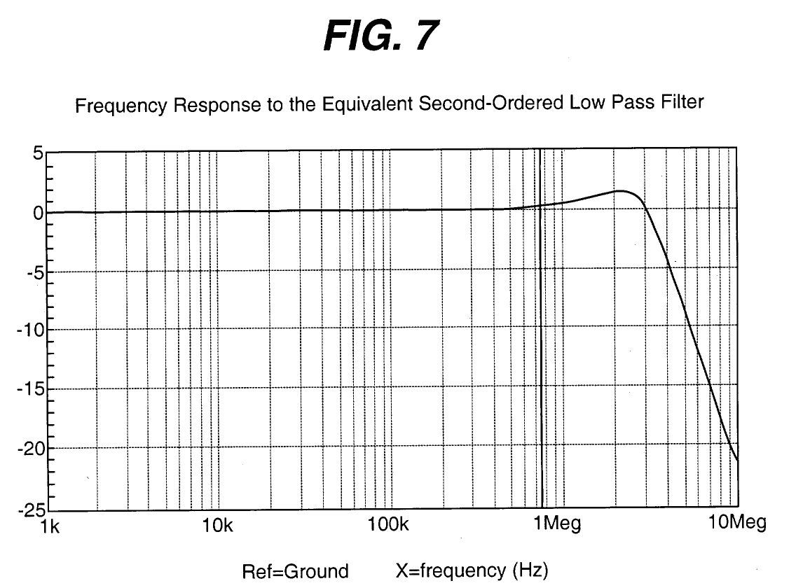

By analyzing a frequency response to the circuit provided in FIG. 6, it can be shown that the natural roll-off point of the low pass filter is at approximately 2.89 MHz. These values were determined by using a production transformer and subjecting the transformer to several frequencies from a signal generator and performing several calculations to determine the value of the equivalent circuit. A frequency response to the circuit is provided in FIG. 7. As can be observed in FIG. 7, all frequencies above the 2.89 MHz threshold are attenuated at a rate of −40 dB per decade. This is a typical characteristic of a low pass filter made up of two reactive elements. If the load (1000 ohms) is increased it can be shown that the gain at the resonant frequency increases. If the load (1000 ohms) is decreased it can be shown that the gain at the resonant frequency subsequently decreases.

Low Pass Filter using the Output Transformer

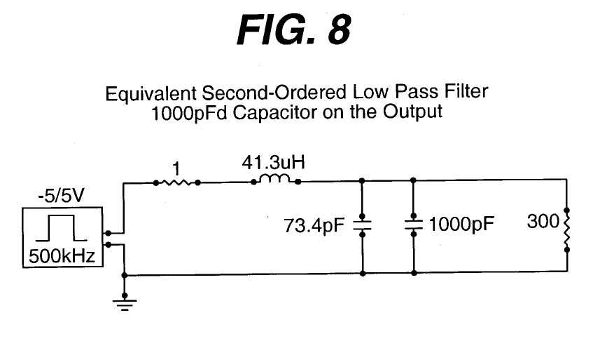

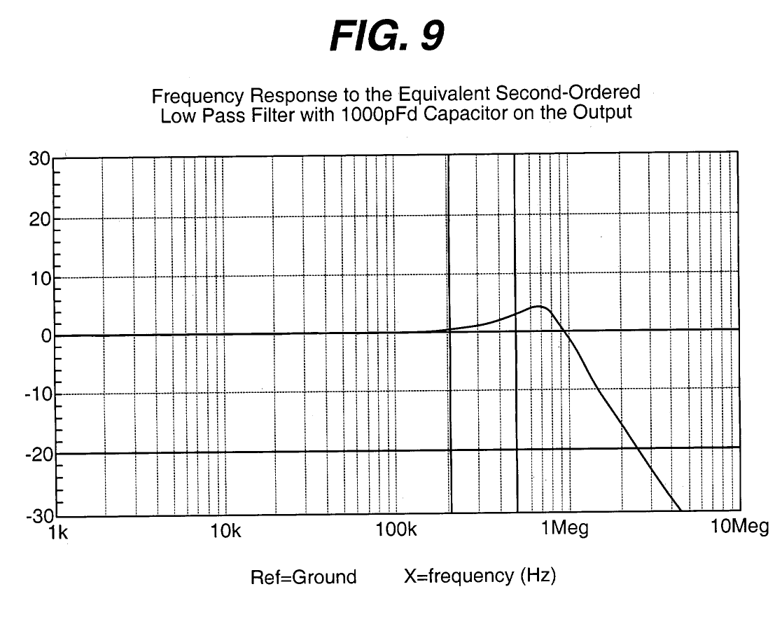

Since the 2.89 MHz threshold is a little too high for the filtering to operate properly, a simple high voltage capacitor is added to the output of the equivalent circuit. Since the resonant frequency is inversely proportional to the size of the capacitance, a capacitor can be added to the output of the transformer and the resonant frequency will drop. By observing the frequency response provided in FIG. 1, we would like to move the resonant point back to approximately 800 kHz. In order to move the frequency back, several capacitors were placed on the output of the circuit. It was determined that a 1000 pFd capacitor added to the output of the transformer, the resonant point moved back to approximately 755 kHz. (The 1000 pFd capacitor was used due to its wide commercial availability.) As shown in FIGS. 8 and 9, the new equivalent circuit and the frequency response can be observed. The gain at the resonant point is starting to get high. In the actual transformer, the resistances in the wires and the core reduce the gain and this “overshoot” is not as high, therefore the filtering characteristics will match the high frequency characteristics of the band pass filter more closely when all of the variables are considered.

High Pass Filter using the Output Transformer

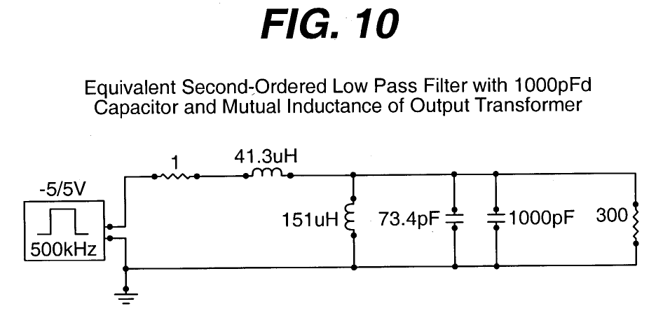

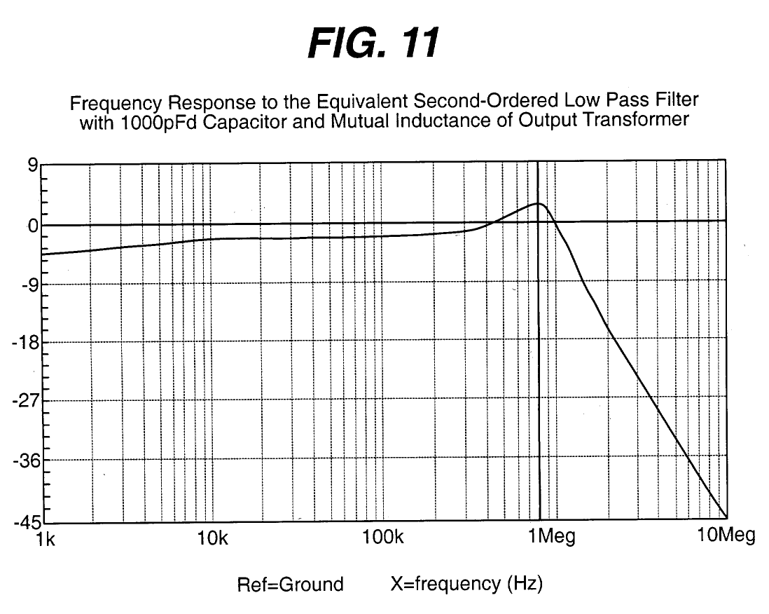

Since the bandpass filter provides attenuation for the low frequencies along with the high frequencies we must achieve similar results with the output transformer. As mentioned before, the mutual inductance can provide some of the needed attenuation for the low frequencies and pass all of the high frequencies. As indicated in FIG. 4, the mutual inductance is shown across the output of the transformer and provides low impedance to all low frequency components. The values of the inductance can vary depending on the desired attenuation. For the present electrosurgical generator, the value of the inductance is 151H. The circuit that produces this frequency response is provided in FIG. 10. For the response of the added mutual inductance refer to FIG. 11.

As seen the FIG. 11, the mutual inductance begins to attenuate the low frequencies but the higher frequencies (between 10 kHz and 100 kHz) are not attenuated except for the reactive divider of the leakage inductance and the mutual inductance. The real transformer has the mutual inductance between the primary and secondary leakage inductance. This will dramatically reduce the effects of the reactive divider of the leakage inductance as well as the mutual inductance. The area in which the attenuation is shown is from 1 kHz to 10 kHz.

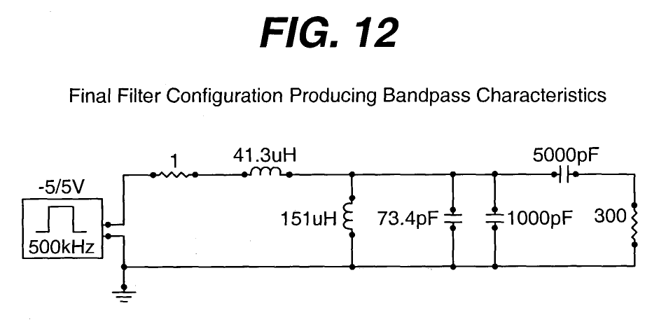

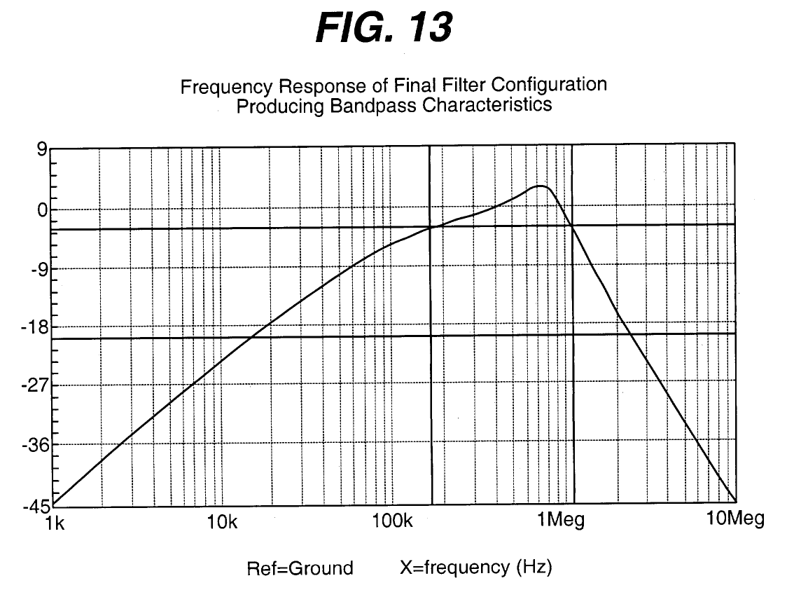

The amount of attenuation needs to increase in order to be close to the bandpass filter attenuation shown in FIG. 1. In order to do this, a capacitor will be added to the output in series with the output load. This capacitor is common in all electrosurgical generators and is required by many test and regulatory agencies. In the present electrosurgical generator the capacitors are added to both leads that go to the patient, one on the positive lead and one on the negative lead. This provides an increased safety margin and ensures that the low frequency attenuation is high. Each of the capacitors has a value of 10,000 Fd, so the series equivalent of the two capacitors is approximately 5,000 Fd. The circuit provided in FIG. 12 shows the final configuration of the filter using the transformer as part of the bandpass filter. When comparing the frequency response illustrated in FIG. 13 and the frequency response provided in FIG. 1, similar results are observed. In both cases both the high and low frequencies are blocked and the fundamental frequencies are passed. The “pass-band” frequencies range from approximately 160 kHz to 1.1 MHz. This is a wider bandwidth than the original bandpass filter but is adequate for the requirements of the electrosurgical generator.

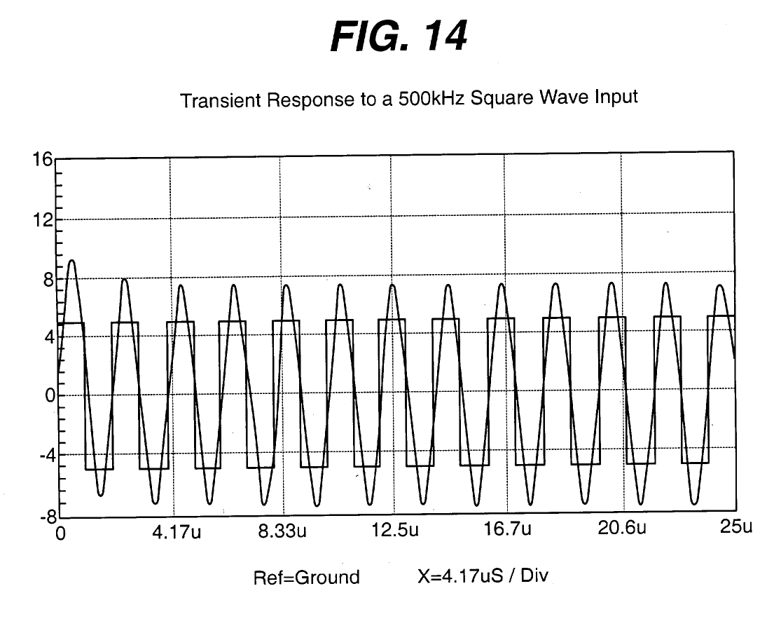

By comparing the waveforms provided in FIGS. 3 and 14, it is shown that the two waveforms are very similar. In both cases the square wave input is converted to a “clean” sine wave. This produces the appropriate output waveform to create good clinical effect while lowering the RF energy in the high frequencies above the fundamental thus reducing the effect of high RF leakage.

Transformer Simulation

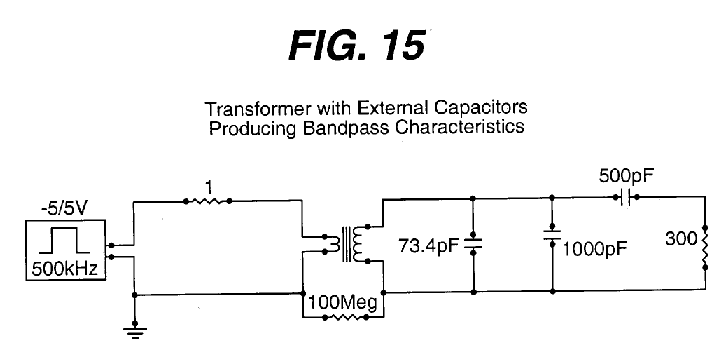

The circuit provided in FIG. 15 replaces the equivalent components with the actual high frequency transformer. The simulation program does not allow for a stray capacitance to be applied to the transformer model. Therefore, adding 73.4 Fd of stray capacitance to the model is still required. The leakage inductance is accounted for through the “K Factor” of the transformer. In this case the “K Factor” is approximately 0.89.

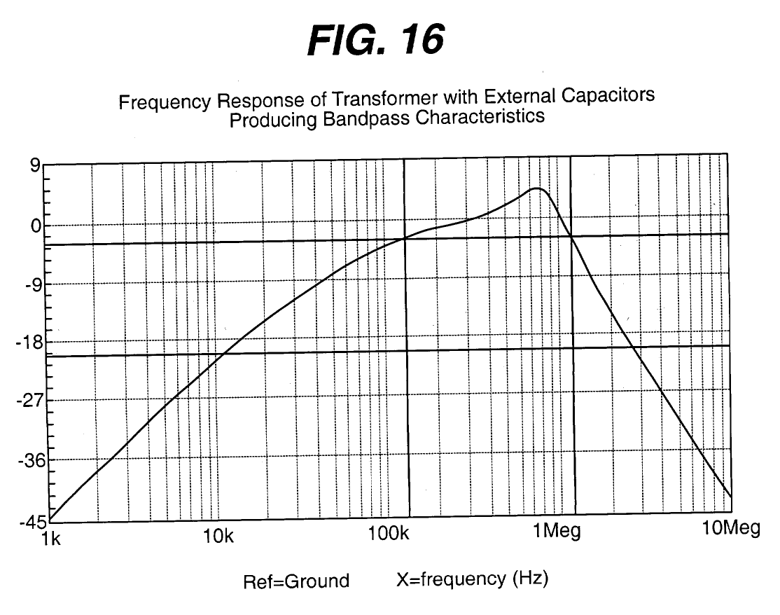

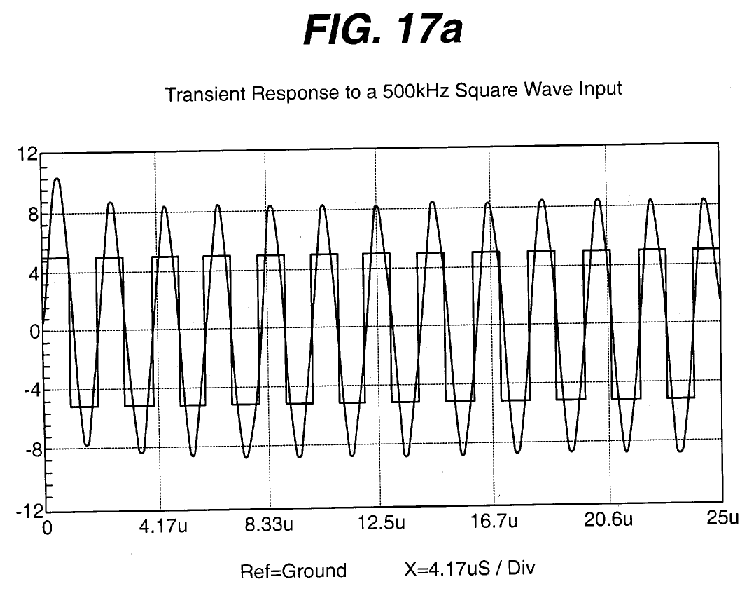

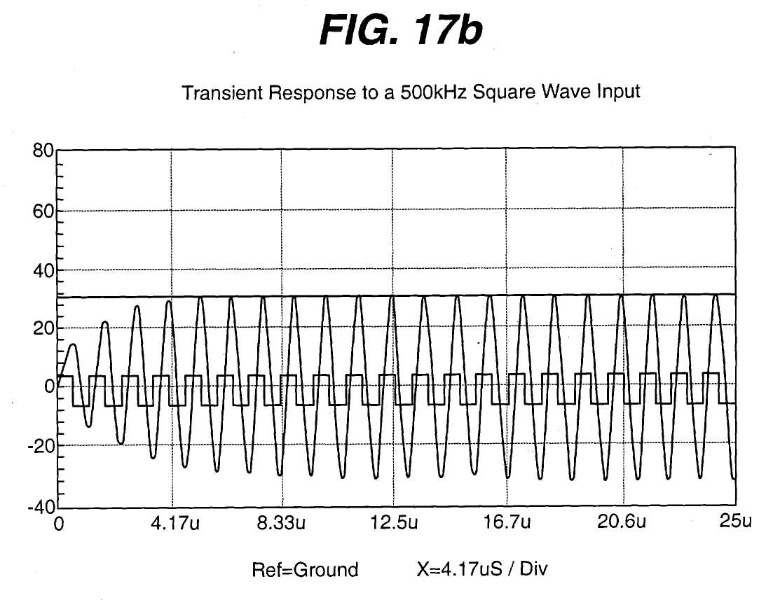

As can be seen in the frequency response of the transformer with the added capacitors in FIG. 16, the frequencies around 500 kHz are passed while both the low and high frequencies are attenuated. By observing the graph, the two frequencies that cross the −3 db points are approximately 128 kHz and 1.19 MHz. By subtracting the lower frequency from the higher frequency the bandwidth of the filter in FIG. 15 can be determined. In this case the bandwidth is approximately 1 MHz. Stating this another way; all frequencies between 128 kHz and 1.19 kHz are passed to the output load (in the case of electrosurgery; the patient) while all frequencies outside of these frequencies are considered to be in the stop bands and are prohibited from going to the output load. The bandwidth can be adjusted by changing the values of the 1000 pFd and the 5000 pFd capacitors. This bandwidth, though larger than the original bandwidth of the bandpass filter in FIG. 1, is adequate for the present electrosurgical generator. As a result, a square wave waveform, operating at approximately 500 kHz, subjected to the filter would produce a sine wave waveform since the fundamental frequency would pass and all high and low frequencies will be stopped. This is shown in the transient response provided in FIGS. 17a and 17b.

Voltage Gain as a Result of Frequency Response

One added benefit to the frequency response as shown in FIG. 16 is the voltage gain at certain frequencies. For example, if the operating frequency was adjusted to approximately 760 kHz, a voltage gain of roughly 4.5 dB could be realized with a load impedance of 300, shown in FIG. 17a. In some modes of operations, such as Spray Coagulation, a high open circuit voltage is required. To illustrate this the load impedance was increased to 1000 and the frequency was adjusted to a point that matched the peak of the resonant point with the new load resistor. As a result, the frequency was adjusted to approximately 840 kHz. When the same 5-volt square wave is subjected to the transformer the voltage increased to approximately 31 volts peak, shown in FIG. 17b. This provides a method to increase open circuit voltage without increasing the transformer turns ratio.

In conclusion, by taking advantage of the transformer's inductive characteristics and applying very specific values of capacitance in certain locations, a bandpass filtering characteristic can be achieved producing a sinusoidal output waveform when subjected to an input voltage of a square wave. The square wave input voltage allows the designer to switch the RF amplifier in a very efficient class D amplifier configuration. Since the transformer is now part of the bandpass filter, high cost inductors are not needed as they are in the separate bandpass filter. In addition, by adjusting the fundamental frequency of the system the voltage gain can be changed. This allows for lower turns ratio to be used in applications where high open circuit voltages are required.

It will be apparent from the foregoing that, while particular forms of the invention have been illustrated and described, various modifications can be made without departing from the spirit and scope of the invention. Accordingly, it is not intended that the invention be limited, except as by the appended claims.

I claim:

1. An electrosurgical generator for supplying radio frequency (RF) power to an electrical instrument, the generator comprising an RF output stage having an output transformer for the delivery of RF power to the instrument, a power supply for supplying power to the output stage and an output bandpass filter comprising the output transformer.
